(19) United States
(12) Patent Application Publication
(10) Pub. No.: US 2015/0376228 A1
(43) Pub. Date: Dec. 31, 2015

(54) PROCESS FOR HIGH EFFICIENCY REFOLDING OF RECOMBINANT PROTEINS

(71) Applicants: Archana KRISHNAN, THANE (IN); Sanjay SONAR, THANE (IN); NIKHIL GHADE, MUMBAI (IN); FAIZA SHAIKH, (IN)

(72) Inventors: Archana KRISHNAN, THANE (IN); Sanjay SONAR, THANE (IN); NIKHIL GHADE, MUMBAI (IN); FAIZA SHAIKH, (IN)

(73) Assignee: BioGenomics Limited, Thane West, MH (IN)

(21) Appl. No.: 14/768,036

(22) PCT Filed: Feb. 21, 2014
(86) PCT No.: PCT/IN2014/000111

§ 371 Date: Aug. 14, 2015

(30) Foreign Application Priority Data
Feb. 22, 2013 (IN) 512/MUM/2013

(51) Int. Cl. C07K 1/113

(52) U.S. Cl. CPC C07K 1/113

(57) ABSTRACT

A process of refolding a recombinant protein from inclusion bodies (IBs) formed inside a host cell, is provided. The process includes homogenising a wet cells slurry to obtain a cell lysate; incubating the cell lysate with a reducing agent to obtain a reduced cell lysate; isolating reduced IBs from the reduced cell lysate to obtain isolated IBs; solubilising the isolated IBs with a denaturing agent to obtain solubilised IBs; and subjecting the solubilised IBs to a unit process of refolding to obtain a refolded recombinant protein.

### CROSS REFERENCE TO RELATED APPLICATION

This application claims priority to the pending PCT application PCT/IN2014/000111, filed on 21 Feb. 2014. The pending PCT application PCT/IN2014/000111 is hereby incorporated by reference in its entireties for all of its teachings.

### FIELD OF THE INVENTION

The present invention relates to production of recombinant proteins, and more particularly to refolding of recombinant proteins from inclusion bodies produced in prokaryotic host cells.

### DESCRIPTION OF THE RELATED ART

Recombinant DNA (rDNA) technology has been used to clone, express and purify several proteins of therapeutic or other economic value such as Insulin, Insulin analogues, trypsin, Granulocyte Colony Stimulating Factor (G-CSF), Granulocyte Macrophage Colony Stimulating Factor (GM-CSF), etc., from prokaryotic as well as eukaryotic cells. However, the use of prokaryotic cells e.g. E. coli is more widespread owing to the better cost-benefit economics of production of recombinant proteins. E. coli bacteria or other prokaryotic host cells are easy to cultivate, since they are capable of producing biomass at a rapid rate. This enables their use in high-cell density fermentations with much better scalability than eukaryotic host cell based fermentations or cell cultures.

The above economic advantages are, however, challenged by the fact that E. coli are unable to perform post-translational modifications of the proteins that are produced in them. Further, due to increased expression in E. coli, the recombinant proteins have proclivity to aggregate as inclusion bodies. Inclusion bodies (IBs) are the aggregates of insoluble, biologically inactive and unfolded proteins that are produced intracellularly in bacteria. IBs also include the protein of interest or the target protein e.g. Insulin, Insulin analogues, trypsin, Granulocyte Colony Stimulating Factor (G-CSF), Granulocyte Macrophage Colony Stimulating Factor (GM-CSF), etc. It is from IBs that the concentrated target protein is purified. After isolation from the host cell proteins (HCPs), the target protein is refolded or renatured to its biologically active form or conformation. A modest increase in yield of biologically active proteins may lead to substantial commercial benefits. The problems that are usually encountered during the renaturation, isolation and purification of the biologically active recombinant protein include misfolding of proteins, protein loss, protein aggregation etc. This is further complicated by the fact that the traditional processes of obtaining biologically active refolded recombinant proteins are multi-step process that include treating of inclusion bodies through a number of reagents and subjecting them to a series of processes such as centrifugation, filtration, dialysis etc. This leads to a great amount of protein loss leading to lower yield of biologically active recombinant protein.

Traditionally, recombinant proteins are obtained in biologically active form by a process that includes lysing of cells for isolating inclusion bodies through centrifugation of lysed cell solution. Thereafter, the isolated inclusion bodies are reconstituted in a buffer having a number of additives, denaturing agents, reducing agents, etc. Following the treatment of IBs with the buffer, the buffer and its components are removed through process of Diafiltration, after which, the IBs are subjected to a unit process of refolding through one of the number of methods that are currently available e.g., oxidation method, sulphonation based methods, etc. The refolded protein is obtained in diluted form, which is concentrated by ultrafiltration and other relevant filtration techniques. The process of refolding is often governed by pH of the buffers, concentration of the additives, reducing agents, redox agents, denaturing agents used etc.

Refolding of recombinant proteins is a highly complex process to optimise for higher yield of correctly refolded recombinant protein. This is mainly due to the conflicting environments that are required throughout the process of refolding, starting from cell lysing. As mentioned, before, as soon as inclusion bodies (IBs) are isolated after lysis of cell, they are directly added to a dissolving buffer or solution having a reducing agent as well as a chaotropic (denaturing) agent, along with a number of additives such as EDTA and thereafter, the dissolved or solubilised IBs are subject to refolding conditions for obtaining a properly folded protein. However, presence of reducing agents and chaotropic agents necessitates the removal of them prior to refolding. As mentioned, this is usually done by Diafiltration or other processes that lead to desalting. The current body of prior art, though attempts to optimise the refolding of protein through variations in reaction conditions, reactants and overall process parameters, but still cannot addresse the inherent paradoxes or conflicts that lead to lower yield of correctly refolded recombinant protein. As such, the current processes are not optimised to address the paradoxes involved in process of refolding of recombinant protein. These paradoxes may pertain to applying different reaction conditions that may be required at different steps of the process to obtain refolded recombinant protein. The three main paradoxes are reduction paradox, dilution paradox and denaturant paradox. Reduction paradox implies that though reduction of recombinant proteins in IBs is preferred to be done at high pH conditions but high pH also leads to aggregation of IBs. Dilution paradox implies that unit process of refolding of recombinant protein is, though, preferred at infinite dilution, the volume of the buffers during the process should be kept to minimum for efficient refolding. Denaturant paradox implies that though denaturant must be present before the unit process of refolding, but it is also required in very low concentrations during the unit process of refolding too.

Many attempts have been made to optimise refolding of recombinant proteins in terms of optimising pH, optimising buffer content, but only a fewer exist that reduce the number of steps, required for refolding recombinant proteins, to a minimum possible. The attempts in which the number of steps have been reduced often comprise use of complex buffers and more additives, thereby impacting the overall process economics in a negative manner. Since, more the additives more are the number of steps or more is the duration of time required to remove them for obtaining purified, concentrated and renatured recombinant protein. Despite several attempts to obtain high efficiency refolding, none of the attempts have been directed towards removal of inherent conflicts or paradoxes that exist within the different method steps in the process of obtaining a refolded recombinant protein. As such, the existing attempts are highly protein specific and lack universal approach towards refolding of recombinant proteins that are obtained from prokaryotic host cells e.g. E. coli. Accordingly, there is a need for a process, with minimum possible steps or stages, for obtaining refolded proteins with higher yields in economically significant manner.

### SUMMARY OF THE INVENTION

In view of the foregoing, the embodiments herein, provide a process for refolding of recombinant proteins that optimises the yield by providing optimal reducing, isolation, solubilisation and refolding conditions.

In an aspect, a process of refolding a recombinant protein isolated from inclusion bodies (IBs), formed inside host cells is provided. The process includes homogenising a wet cells slurry of the host cells to obtain a cell lysate; wherein the cell lysate includes IBs; incubating the cell lysate with a reducing buffer to obtain a reduced cell lysate; isolating reduced IBs from the reduced cell lysate to obtain isolated reduced IBs; solubilising the isolated reduced IBs with a denaturing agent to obtain reduced solubilised IBs; and subjecting the reduced solubilised IBs to a unit process of refolding to obtain a refolded recombinant protein.

In another aspect, a process of refolding a recombinant protein from inclusion bodies (IBs) formed inside host cells is provided. The process includes homogenising a wet cells slurry to obtain a cell lysate; wherein the cell lysate includes IBs; incubating the cell lysate with a reducing agent, at a pH ranging between 7.0 and 9.0, and preferably at 8.0, to obtain a reduced cell lysate; diluting the reduced cell lysate with at least 20 volumes buffer at pH ranging between 7.3 and 7.5, to obtain a diluted cell lysate; isolating reduced IBs from the diluted cell lysate to obtain isolated IBs at pH ranging between 7.3 and 7.5; reducing pH of the isolated IBs to 3.0; solubilising the isolated IBs at pH 3.0 with a denaturing agent at pH ranging between 2.5 and 4.0, to obtain acidic pH solubilised IBs; and diluting the acidic pH solubilised IBs with a continuously flowing refolding buffer at pH ranging between 7.5 and 11.5 to obtain a refolded biologically active recombinant protein, such that when the solubilised IBs are introduced into the continuous flow of refolding buffer, concentration of the denaturing agent is reduced to preferably lesser than 0.3 M but not more than 3 M, and concentration of the recombinant protein in solubilised IBs is in range of 0.1 g/L to 1 g/L.

### BRIEF DESCRIPTION OF THE DRAWINGS

For a more complete understanding of the embodiments herein, reference should now be made to the embodiments illustrated in greater detail in the accompanying drawings and described below by way of examples:

FIG. 1 is a flow chart illustrating process of refolding of recombinant proteins, according to an embodiment herein; and

FIG. 2 is a bar chart illustrating effect of presence of reducing agent, on protein refolding, during inclusion body isolation, according to an embodiment herein; and

FIG. 3 is a bar chart illustrating effect of treating the solubilised inclusion bodies in form of pellet with a denaturing agent on protein refolding, according to an embodiment herein.

### DETAILED DESCRIPTION OF THE INVENTION

As required, detailed embodiments are disclosed herein; however, it is to be understood that the disclosed embodiments are merely exemplary, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language).

As mentioned, there is a need to develop a process, with minimum steps or stages along with minimal use of additives, for obtaining refolded proteins with higher yields in economically significant manner. As mentioned before, as soon as the cells are lysed to isolate inclusion bodies, the proteins are oxidised due to their exposure to atmosphere, leading to formation of incorrect disulfide bonds, leading to lower yield of refolded proteins.

Recombinant proteins may be produced in prokaryotic or eukaryotic host cells. The environment of a cell, such as E. coli cell is reducing in nature, therefore, the current embodiments provide a process that includes maintaining a reducing environment while isolation of a precursor (in form of inclusion bodies) essential for achieving quantitative folding of the recombinant protein as described herein. The recombinant protein is the target protein for isolation, refolding and purification and may be selected from Insulin and Insulin analogues, G-CSF and GCSF analogues, GM-CSF and GM-CSF analogues, monoclonal antibodies such as trastuzumab, etanercept, bevacizumab etc., interferons, erythropoietin, human growth hormone, trypsin, carboxypeptidase and many other recombinant proteins or peptides of therapeutic or non-therapeutic significance.

The embodiments herein identify the major conflicts or paradoxes in the process of refolding of recombinant proteins and provide a process that addresses the three paradoxes of reduction, dilution and denaturant. It has been observed that different pH buffers or solutions are required for dissolving IBs than subsequent denaturing or refolding so as to attain an optimal yield of a recombinant protein with minimal usage of additives. It has also been observed that isolating inclusion bodies in presence of a reducing agent in conditions of basic pH leads to greater yield of correctly refolded protein. To this effect, the embodiments herein, provide a process for refolding of a recombinant protein, in which the inclusion bodies are isolated in presence of a reducing agent at basic pH conditions, and then subjected to denaturation in acidic pH conditions in presence of a denaturing agent, followed by refolding in basic pH conditions in which no additional denaturing agent is added. In the embodiments herein, the process of reduction of inclusion bodies, post cell-lysis, is performed at basic pH and that of denaturation of reduced inclusion bodies (obtained after isolation in presence of a reducing agent), is performed at an acidic pH leading to separation of the two processes leading to higher yield of correctly folded recombinant protein.

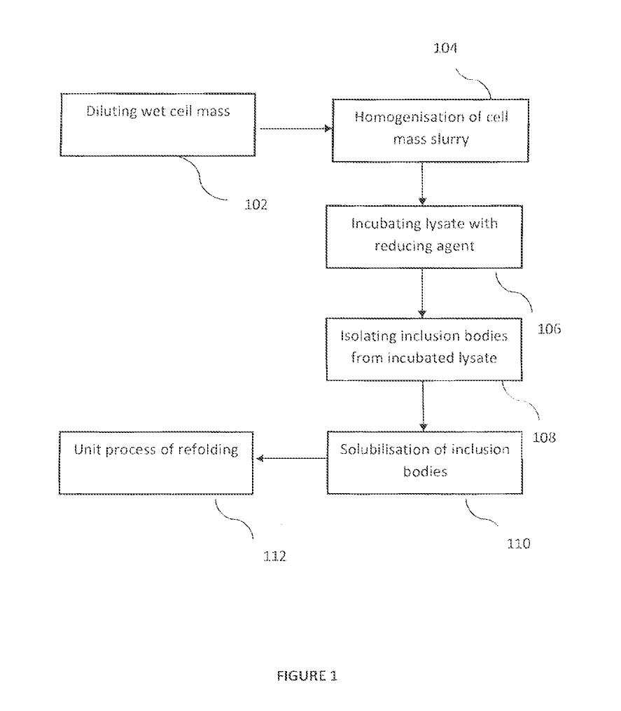

FIG. 1 is a flow chart illustrating process of refolding of recombinant proteins, according to an embodiment herein. The process includes obtaining wet cells and diluting it to obtain wet cell slurry in step 102. Thereafter, the slurry is subjected to homogenisation by cell lysis to obtain a lysate, in step 104. In one embodiment, the cells in the slurry are lysed by cell disruptors or sonicators. The lysate includes inclusion bodies and lysed cells. Further, in step 106, the lysate is incubated with a reducing agent for a fixed duration. Reducing agent may be selected from a group consisting of, but not limiting to, DTT, β-mercaptoethanol, cysteine, dithioerythrito (DTE), cysteamine, thioglycolate, glutathione, or sodium borohydride. In one embodiment, reducing agents are cysteine blocking agents. The concentration of reducing agent is in range of 0.1 mM to 10 mM at a pH of 7.0 to 9.0. In a preferred embodiment, the concentration of reducing agent is 0.25 mM. The preferred pH for reducing conditions is 8.0. Alternatively, the lysate may also be treated with a reducing buffer. The reducing buffer includes a reducing agent, a chaotropic agent and a salt at optimal concentrations along with known additives.

The lysate incubated with the reducing agent or the reducing buffer is treated, in step 108, to isolate inclusion bodies in form of a pellet or slurry. According to the one embodiment, the inclusion body isolation is done in presence of a reducing agent. In another embodiment, the reducing agent is introduced, in protein refolding process, before isolation and solubilisation of inclusion bodies so as to maintain a cell-like reducing environment throughout the critical steps of process of refolding recombinant protein. In yet another embodiment, before inclusion body isolation, the reduced lysate is diluted with 20× volumes of a buffer at a neutral pH or at a pH in range of 7.3 to 7.5. The isolation of the reduced and diluted lysate in step 108 removes remaining amount of reducing agent and other contents (if the reducing buffer is used). The pellet obtained in the step 108 is at a pH of 7.3 to 7.5. The reduced inclusion bodies may be isolated from the reduced lysate from methods consisting of, but not limiting to centrifugation, ion exchange chromatography, affinity chromatography, diafiltration, reverse phase chromatography, chromatography, precipitation, etc. In a preferred embodiment, the reduced inclusion bodies are isolated from the reduced lysate in a continuous centrifuge. In yet another preferred embodiment, the reduced inclusion bodies are isolated from the reduced lysate in a batch centrifuge.

The pellet obtained in the step 108 is reconstituted with a chaotropic agent, in step 110, for solubilisation at acidic pH. The pH of the chaotropic agent or denaturing agent may be reduced using a strong acid. Chaotropic agent may be selected from group consisting of, but not limiting to, urea, guanidine, arginine, sodium thiocyanate, SDS, sarkosyl, chlorides, nitrates, thiocyanates, cetylmethylammonium salts, trichloroacetates, chemical solvents such as DMSO, DMF) or strong anion exchange resins such as Q-Sepharose. Alternatively, a solubilising buffer may be formed. The solubilising buffer includes a denaturing agent and a buffering agent at an acidic pH. In one embodiment, the pH for solubilisation is in range of 2.5 to 4.0. In one embodiment, the pH of the pellet obtained in step 108 is immediately adjusted to an acidic pH in range of 2.5 to 4.0, and centrifuged to obtain a pellet cake. Alternatively, slurry of the reduced and solubilised inclusion bodies may be obtained.

The pellet cake is then reconstituted with chaotropic agent at acidic pH or a solubilising buffer at acidic pH. In one embodiment, the solubilising buffer includes the chaotropic agent. The reconstitution of the pellet or the pellet cake is done in such a way that the concentration of reconstituted or solubilised inclusion bodies is in range of 15-18 g/L. Absolute solubilisation of inclusion bodies may be obtained using a high speed blender. Additionally, to achieve absolute solubilisation, the reduced and solubilised inclusion bodies may be exposed briefly to a conditions or environment with pH ranging between 7.0 and 11.0, before adjusting the pH of the solubilised inclusion bodies back in the range of 2.5 and 4.0, preferably at 3.0.

The solubilised inclusion bodies are then subjected to unit process of refolding in step 112. In one embodiment, the unit process of refolding is based on dilution refolding. In step 112, the solubilised inclusion bodies are subjected to treatment with a refolding buffer. The refolding buffer may include a buffering agent such as Sodium bicarbonate and a chelating agent such as EDTA at a basic pH. In one embodiment the refolding buffer does not include any chelating agent. In yet another embodiment, the refolding buffer is deionised water maintained at a pH in range of 7.5 to 11.5. In a preferred embodiment, refolding buffer is any buffer or solution that is used in the unit process of refolding to obtain correctly folded recombinant protein. In one embodiment, the pH of refolding buffer is in range of 7.5 to 11.5, and more preferably at 10.5.

The volume of refolding buffer used for dilution is calculated to obtain a concentration of the recombinant protein in inclusion bodies is in range of 0.1 g/L to 1 g/L. In one embodiment, the final concentration of the recombinant protein in the inclusion bodies in the refolding buffer is adjusted to 0.4 g/L. In another preferred embodiment, the final concentration of the recombinant protein in the inclusion bodies in the refolding buffer is adjusted to 0.1 g/L. The final concentration of urea in the refolding buffer is adjusted such that it is not more than 3 M, but preferably less than 0.3 M. In one embodiment, the final concentration of urea in the refolding buffer is adjusted to less than 0.3 M. The refolding mixture includes refolding buffer and the solubilised inclusion bodies.

The pH of the refolding mixture is maintained in the range of 7.5 to 11.5. In one embodiment, the pH of the refolding mixture is maintained at 10.5. Further, the process of refolding is carried out in presence of atmospheric air which is introduced in the refolding mixture in form of bubbles through a number of spargers. The pressure of air is range of 0.01 bar to 2.5 bar. The temperature is maintained in the range of 4° C. to 25° C.

In one embodiment, a very low amount of solubilised inclusion bodies are introduced into a stream of continuous buffer flow in such a way that when introduced into the stream the resultant concentration of the target protein or the recombinant protein in the inclusion bodies is in range of 0.1 g/L-1 g/L and that of Urea is not more than 3 M, preferably less than 0.3 M. In one embodiment, the solubilised IBs are introduced into the refolding buffer in continuous flow arrangement such that concentration of the denaturing agent in the solubilised IBs is reduced below a concentration that is required for denaturing the IBs and, thereafter, the recombinant protein in the solubilised IBs is refolded into a biologically active refolded recombinant protein. This method saves on using large volumes of buffer or water. In one embodiment, the process of refolding may be carried out using deionised water.

In the process of refolding of the proteins, described above, no chaotropic agent is added during the unit process of refolding in the refolding mixture. The residual urea from the solubilisation of inclusion bodies is used to refold the proteins (in inclusion bodies) during the unit process of refolding. Further, no reducing agent is required in the refolding buffer, during the unit process of refolding. The process of refolding of recombinant protein as described herein includes the process starting from homogenisation of cell slurry to refolding of the recombinant protein in the unit process of refolding. According to the embodiments herein, the unit process of refolding includes the various methods applied to obtain refolded recombinant protein such as infinite dilution method, oxidation method etc. The concentration of the recombinant protein in IBs, or in any solution or buffer may be determined by commonly known protein estimation methods such as Bradford estimation, Laurie estimation, using extinction coefficient of the protein of interest in the UV range etc. The examples given below in a non-limiting manner will make it possible to better understand the embodiments herein.

### EXAMPLES

### Example 1

### Homogenisation of Wet Cells

After fermentation, the wet cells were harvested using a continuous centrifuge. The wet cells were diluted with 20× volumes of 20 mM Tris. The 20× volume of the Tris buffer was calculated on the basis of theoretical pellet weight of the wet cell mass. The slurry of the wet cells, obtained after dilution, was subjected to homogenisation by lysing cells using a cell disruptor up to 3 passes. The lysate obtained after the cell lysis contained inclusion bodies and lysed cells. The lysate was incubated with 1 M Urea, 150 mM Sodium Chloride, and 0.25 mM DTT at 25° C. for 1 hour.

### Example 2

### Inclusion Body Isolation

The DTT reduced lysate was diluted with 20× volumes of phosphate buffer saline at a pH range of 7.3 to 7.5. The diluted lysate was subjected to centrifugation at 14000 G-15000 G force using a Westfalia continuous centrifuge at a feed flow rate of 20-22 litres per hour. The resulting supernatant (S1) and pellet (P1) were collected separately. P1 was washed with phosphate buffered saline by centrifugation at 14000 G-15000 G force using the Westfalia continuous centrifuge. The resulting supernatant (S2) and the pellet (P2) was used for further inclusion body solubilisation.

### Example 3

### Inclusion Body Solubilisation

The pellet (P2) slurry that was obtained after centrifugation was at a pH in the range of 7.3 to 7.8. The pH of the slurry was immediately adjusted pH in the range of 2.5 and 4.0 using Hydrochloric Acid (HCl), The slurry at low pH was centrifuged using a batch centrifuge at 6000 rpm for 15 minutes Inclusion bodies in the form of a compact pellet cake were obtained. The pellet was reconstituted in 8M urea, 2.5 mM glycine at pH ranging between 2.5 and 4.0. The volume of the reconstituting buffer was calculated such that the recombinant protein in the inclusion bodies had the final concentration of 15-18 g/L. The inclusion body pellet was completely solubilised using a blender.

### Example 4

### Unit Process of Refolding

The solubilised inclusion bodies were used as refolding load sample and the refolding was carried out using dilution techniques. The solubilised inclusion bodies were treated with the refolding buffer having 10 mM sodium bicarbonate, 1 mM EDTA at pH of 10.5. The volume of the buffer was calculated in a way to achieve a final concentration in range of 0.1 g/L-1 g/L of the inclusion bodies, whereas the concentration of urea is maintained at not more than 3.0 M, preferably lesser than 0.3 M. The pH of the refolding mixture was maintained at 10.5, with the refolding process carried out at 20° C.-25° C. in presence of oxygen at atmospheric pressure. The atmospheric air was introduced to the refolding mixture in form of bubbles through spargers.

### Example 5

### Effect of Inclusion Body Isolation, in Presence of Reducing Agent, at Basic pH on Amount of Refolded Protein Obtained

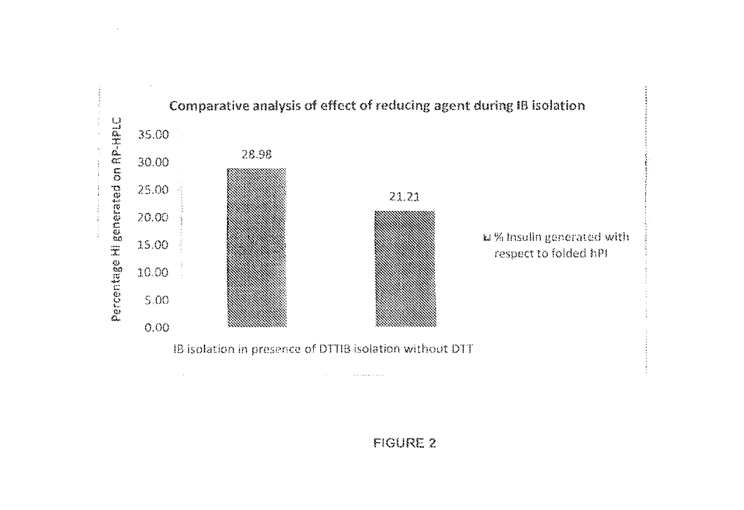

The effect of inclusion body isolation in presence of reducing agent was analysed, as illustrated in FIG. 2. Refolding was monitored on RP-HPLC. The refolded monomeric human insulin precursor was enzymatically digested to obtain active insulin. The amount of insulin generated by the two methods of inclusion body isolation i.e. with and without the presence of reducing agent; was determined. As may be observed in FIG. 2, the amount of insulin obtained through the process of inclusion body isolation in presence of a reducing agent (as per embodiments herein) is more (at 28.98%) than the amount of insulin obtained through the process of inclusion body isolation in absence of a reducing agent (at 21.21%).

### Example 6

### Effect of Solubilised Inclusion Body Treatment, with Urea, at Acidic pH on Amount of Refolded Protein Obtained

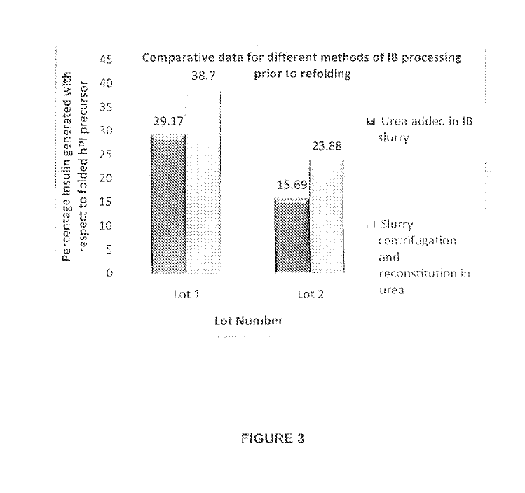

The solubilised inclusion bodies were treated with 8 M urea in form of pellet cake (as per embodiments herein) and as slurry. FIG. 3 illustrates the effect of treating the solubilised inclusion bodies in form of pellet with a denaturing agent on protein refolding. The amount of insulin (38.7%) generated by treating solubilised inclusion bodies in pellet form/state with 8M urea was more than the amount of insulin (29.17%) generated by treating solubilised inclusion bodies in slurry form with 8M urea.

We claim:

1. A process of refolding a recombinant protein isolated from inclusion bodies (IBs), formed inside host cells, comprising:
(a) homogenising a wet cells slurry to obtain a cell lysate; wherein said cell lysate comprises IBs;
(b) incubating said cell lysate with a reducing buffer to obtain a reduced cell lysate; said reduced cell lysate comprising reduced IBs;
(c) isolating said reduced IBs from said reduced cell lysate to obtain isolated reduced IBs;
(d) solubilising said isolated reduced IBs with a denaturing agent to obtain reduced solubilised IBs; and
(e) subjecting said reduced solubilised IBs to a unit process of refolding to obtain a refolded recombinant protein.

2. The process of claim 1 further comprises;
diluting said reduced cell lysate at a pH ranging between 7.3 and 7.5; and
adjusting pH of said isolated IBs to a pH ranging between 2.8 and 3.5.

3. The process of claim 1, wherein said reducing buffer consists of a reducing agent only, in a buffer with basic pH, preferably between 7.0 and 9.0.

4. The process of claim 1, wherein said reducing buffer comprises a denaturing agent and a reducing agent in a buffer with basic pH, preferably between 7.0 and 9.0

5. The process of claim 3 or 4, wherein said reducing buffer is at a pH ranging between 7.0 and 9.0. and said reducing agent is at a concentration ranging between 0.1 mM and 10 mM.

6. The process of claim 1, wherein said IBs are isolated in presence of a reducing agent before solubilisation and said denaturing agent is maintained in conditions with pH ranging between 2.5 and 4.0.

7. The process of claim 1, wherein said isolated IBs are obtained in form of a pellet cake that is solubilised or reconstituted in said denaturing agent such that concentration of said recombinant protein in said IBs is in range of 15-18 g/l.

8. The process of claim 1, wherein said cell lysate is incubated with Dithiothreitol (DTT) at 25° C. for 1 hour at pH ranging between 7.0 and 9.0 to obtain said reduced cell lysate.

9. The process of claim 1, wherein said unit process of refolding comprises diluting said solubilised IBs into a refolding buffer at pH ranging between 7.5 and 11.5, to obtain a refolding mixture, such that in said refolding mixture, concentration of said denaturing agent is not more than 3 M and is preferably less than 0.3 M, and concentration of said recombinant protein in said solubilised IBs is in range of 0.1 g/L to 1 g/L in said refolding mixture.

10. The process of claim 8, wherein concentration of said denaturing agent is 0.3 M and concentration of said recombinant protein in said solubilised IBs is 0.4 g/L in said refolding mixture and pH of said refolding buffer is 10.5.

11. The process of claim 8 further comprising oxidation of said refolding mixture through air bubbles introduced through spargers at a pressure ranging between 0.01 bar and 2.5 bar.

12. The process of claim 8, wherein said solubilised IBs are introduced into said refolding buffer in continuous flow arrangement such that concentration of said denaturing agent in said solubilised Ms is reduced below a concentration required for denaturing said IBs and said recombinant protein in said IBs is refolded into a biologically active refolded recombinant protein.

13. A process of refolding a recombinant protein from inclusion bodies (IBs) formed inside host cells, comprising:
(a) homogenising a wet cells slurry to obtain a cell lysate; wherein said cell lysate comprises IBs;
(b) incubating said cell lysate with a reducing agent, at a pH ranging between 7.0 and 9.0, and preferably at 8.0, to obtain a reduced cell lysate; said reduced cell lysate comprising reduced IBs;
(c) diluting said reduced cell lysate with at least 20 volumes buffer at pH ranging between 7.3 and 7.5, to obtain a diluted cell lysate;
(d) isolating reduced IBs from said diluted cell lysate to obtain isolated IBs at pH ranging between 7.3 and 7.5;
(e) reducing pH of said isolated IBs to 3.0;
(f) solubilising said isolated IBs at pH 3.0 with a denaturing agent at pH ranging between 2.5 and 4.0, to obtain acidic pH solubilised IBs; and
(g) diluting said acidic pH solubilised IBs with a continuously flowing refolding buffer at pH ranging between 7.5 and 11.5 to obtain a refolded biologically active recombinant protein, such that when said solubilised IBs are introduced into said continuous flow of refolding buffer, concentration of said denaturing agent is reduced to preferably lesser than 0.3M but not more than 3M, and concentration of said recombinant protein in said solubilised IBs is between 0.1 g/L and 1 g/L in said refolding mixture.

14. The process of claim 13, wherein said reducing buffer is selected from deionised water, a 10 mM sodium bicarbonate buffer or any basic buffer with a pH ranging between 7.5 and 11.5.

15. The process of claim 13, wherein said IBs are completely solubilised by blender or by exposing said acidic pH solubilised IBs briefly to a buffer at basic pH ranging between 7.0 and 11.0, and then adjusting pH of said solubilised IBs back in the range of 2.5 and 4.0, preferably at 3.0
